(19) United States
(12) Patent Application Publication
(10) Pub. No.: US 2026/0207719 A1
(43) Pub. Date: Jul. 23, 2026

(54) METHODS AND PHARMACEUTICAL COMPOSITION FOR THE TREATMENT OF SANFILIPPO SYNDROME TYPE IIIB

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); CENTRE NATIONAL DE LA RECHERCHE SCIENTIFIQUE, Paris (FR); ICM (INSTITUT DU CERVEAU ET DE LA MOELLE EPINIÈRE), Paris (FR); SORBONNE UNIVERSITÉ, Paris (FR); UNIVERSITÉ TOULOUSE III - PAUL SABATIER, Toulouse (FR); CENTRE HOSPITALIER UNIVERSITAIRE DE TOULOUSE, Toulouse (FR); UNIVERSITÉ PARIS-SACLAY, Gif-sur-Yvette (FR); COMMISSARIAT À L'ÉNERGIE ATOMIQUE ET AUX ÉNERGIES ALTERNATIVES (CEA), Paris (FR)

(72) Inventors: Jérôme AUSSEIL, Toulouse Cedex 3 (FR); Françoise PIGUET, Paris (FR); Kumaran DEIVA, Kremelin-Bicetre (FR); Nissrine BALLOUT, Toulouse (FR)

(21) Appl. No.: 19/157,831

(22) PCT Filed: Feb. 20, 2024
(86) PCT No.: PCT/EP2024/054308

§ 371 Date: Aug. 19, 2025

(30) Foreign Application Priority Data
Feb. 21, 2023 (EP) 23305225.7

(51) Int. Cl. A61K 38/47; A61K 48/00; A61P 25/28

(52) U.S. Cl. CPC A61K 38/47; A61K 48/005; A61K 48/0075; A61P 25/28

(57) ABSTRACT

The present invention relates to treatment of the treatment of Sanfilippo syndrome type IIIB. In this study, the inventors developed a new approach by combining intravenous (IV) and intracerebral (IC) injection in the periventricular white matter of a vector AAVPHP.eB encoding NAGLU. They showed that this new approach allows to a good persistence production and release of the therapeutic protein at the injection site and distant tissues for up to 18 months in the absence of immune reaction, induce a little local inflammatory reaction and transduce neurons. Thus, the invention relates to a vector for use in the treatment of Sanfilippo syndrome type B (MPS3B) in a subject in need thereof, which vector comprises the full sequence of N-acetylglucosaminidase, alpha (NAGLU) encoding nucleic acid and wherein the vector is administrated to said subject in the venous system and directly in the brain.

### FIELD OF THE INVENTION

The present invention relates to a vector for use in the treatment of Sanfilippo syndrome type B (MPS3B) in a subject in need thereof, which vector comprises the full sequence of N-acetylglucosaminidase, alpha (NAGLU) encoding nucleic acid and wherein the vector is administrated to said subject in the venous system and directly in the brain.

### BACKGROUND OF THE INVENTION

Mucopolysaccharidosis type IIIB syndrome (also known as Sanfilippo syndrome type B) is a rare autosomal recessive lysosomal storage disorder with predominant neurological manifestation in affected children. It is caused by mutations in the a-N-acetylglucosaminidase (NAGLU) gene, coding for a lysosomal enzyme required for the stepwise degradation of heparan sulfate glycosaminoglycans (GAGs). The accumulation of incompletely degraded GAGs in affected cells and extracellular spaces leads to cognitive retardation and further neurodegeneration of the central nervous system, leading to progressive deterioration of cognitive abilities before the age of 5 years, including language acquisition delay, cognitive delay and/or abnormal behavior, and premature death in the second decade (1-4). The challenge to treat MPS IIIB syndrome lies in the design of a therapy to supply the missing enzyme to the brain as early as possible after birth. Several recent human gene therapy trials for the treatment of neurodegenerative diseases relied on the deposit of adeno-associated virus (AAV) vectors directly into the brain (5-7). In preclinical studies in MPS IIIB mice (8, 9) and dogs (10, 11), beneficial biochemical and neurological effects with intracerebral gene therapy administered via a recombinant AAV vector encoding NAGLU were observed, associated with the release of therapeutic enzyme from transduced brain cells. These results led to the assessment of safety and efficacy of a novel intracerebral therapy in a phase 1/2 uncontrolled clinical trial, in which four children with MPSIIIB syndrome were enrolled to receive intraparenchymal deposits of a recombinant AAV vector serotype 2/5 (rAAV2/5) encoding human NAGLU combined with immunosuppression (ClinicalTrials.gov Identifier:NCT03300453). An intermediate report at 30 months concluded that treatment was well tolerated and induced sustained enzyme production in the brain. Good tolerance, sustained NAGLU production and milder disease in the patient treated at very early stage were confirmed after a 5.5 years follow-up (Deiva et al. Hum Gene Ther. 2021 October; 32(19-20):1251-1259. doi: 10.1089/hum.2021.135).

However, the neurocognitive benefit was in all cases only partial, presumably because enzyme was not delivered to the periphery, leaving meninges and brain capillaries severely affected. Thus, there is still need of well tolerated, safe and efficient method to treat the patient suffering from Sanfilippo syndrome type B.

### SUMMARY OF THE INVENTION

In this study, the inventors developed a new approach by combining intravenous (IV) and intracerebral (IC) injection in the periventricular white matter of a vector AAVPHP.eB encoding NAGLU. They showed that this new approach allows to a good persistence production and release of the therapeutic protein at the injection site and distant tissues for up to 18 months in the absence of immune reaction, induce a little local inflammatory reaction and transduce neurons.

Thus, the invention relates to a vector for use in the treatment of Sanfilippo syndrome type B (MPS3B) in a subject in need thereof, which vector comprises the full sequence of N-acetylglucosaminidase, alpha (NAGLU) encoding nucleic acid and wherein the vector is administrated to said subject in the venous system and directly in the brain.

Particularly, the invention is defined by its claims.

### DETAILED DESCRIPTION OF THE INVENTION

### The NAGLU Vector and Used Thereof

A first object of the invention relates to a vector for use in the treatment of Sanfilippo syndrome type B (MPS3B) in a subject in need thereof, which vector comprises the full sequence of N-acetylglucosaminidase, alpha (NAGLU) encoding nucleic acid and wherein the vector is administrated to said subject in the venous system and directly in the brain.

According to the invention, an administration of the vector in the venous system allows a targeting of both periphery and nervous system and a direct administration in the brain allow a higher targeting of CNS and enough enzyme activity in the affected cells. The double injection (in the venous system and in the brain) allows a significant and surprising increase of NAGLU expression in the white matter (corpus callosum, general white matter, internal capsule), in the caudate and in the putamen compared to a single injection in the venous system for example.

As used herein, the term “Sanfilippo syndrome type B” also known as “mucopolysaccharidosis type IIIB” or “MPSIIIB” or “MPS3B” has its general meaning in the art and denotes a rare autosomal recessive lysosomal storage disease that primarily affects the brain and spinal cord. It is caused by the defect in the catabolism of large sugar molecules called glycosaminoglycans (GAGs, or mucopolysaccharides) in the lysosomes. The MPS3B is caused by mutations in the lysosomal NAGLU gene.

As used herein, the term “NAGLU” for “N-acetylglucosaminidase, alpha” has its general meaning in the art and denotes an enzyme that degrades heparan sulfate (a GAG subtype) by hydrolysis of terminal N-acetyl-D-glucosamine residues in N-acetyl-alpha-D-glucosaminide. Its Entrez reference number is 4669 (SEQ ID NO:1) and its uniProt reference number P54802 (SEQ ID NO: 2). The two sequences described below relate to the human NAGLU.

SEQ ID NO: 1:
atggaggcggtggcggtggccgcggcggtgggggtccttctcctggccggggccgggggcgcggcaggcg
acgaggcccgggaggcggcggccgtgcgggcgctcgtggcccggctgctggggccaggccccgcggccga
cttctccgtgtcggtggagcgcgctctggctgccaagccgggcttggacacctacagcctgggcggcggc
ggcgcggcgcgcgtgcgggtgcgcggctccacgggcgtggcggccgccgcggggctgcaccgctacctgc
gcgacttctgtggctgccacgtggcctggtccggctctcagctgcgcctgccgcggccactgccagccgt
gccgggggagctgaccgaggccacgcccaacaggtaccgctattaccagaatgtgtgcacgcaaagctac
tctttcgtgtggtgggactgggcccgctgggagcgagagatagactggatggcgctgaatggcatcaacc
tggcactggcctggagcggccaggaggccatctggcagcgggtgtacctggccttgggcctgacccaggc
agagatcaatgagttctttactggtcctgccttcctggcctgggggcgaatgggcaacctgcacacctgg
gatggccccctgcccccctcctggcacatcaagcagctttacctgcagcaccgggtcctggaccagatgc
gctccttcggcatgaccccagtgctgcctgcattcgcggggcatgttcccgaggctgtcaccagggtgtt
ccctcaggtcaatgtcacgaagatgggcagttggggccactttaactgttcctactcctgctccttcctt
ctggctccggaagaccccatattccccatcatcgggagcctcttcctgcgagagctgatcaaagagtttg
gcacagaccacatctatggggccgacactttcaatgagatgcagccaccttcctcagagccctcctacct
tgccgcagccaccactgccgtctatgaggccatgactgcagtggatactgaggctgtgtggctgctccaa
ggctggctcttccagcaccagccgcagttctgggggcccgcccagatcagggctgtgctgggagctgtgc
cccgtggccgcctcctggttctggacctgtttgctgagagccagcctgtgtatacccgcactgcctcctt
ccagggccagcccttcatctggtgcatgctgcacaactttgggggaaaccatggtctttttggagcccta
gaggctgtgaacggaggcccagaagctgcccgcctcttccccaactccaccatggtaggcacgggcatgg
cccccgagggcatcagccagaacgaagtggtctattccctcatggctgagctgggctggcgaaaggaccc
agtgccagatttggcagcctgggtgaccagctttgccgcccggcggtatggggtctcccacccggacgca
ggggcagcgtggaggctactgctccggagtgtgtacaactgctccggggaggcctgcaggggccacaatc
gtagcccgctggtcaggcggccgtccctacagatgaataccagcatctggtacaaccgatctgatgtgtt
tgaggcctggcggctgctgctcacatctgctccctccctggccaccagccccgccttccgctacgacctg
ctggacctcactcggcaggcagtgcaggagctggtcagcttgtactatgaggaggcaagaagcgcctacc
tgagcaaggagctggcctccctgttgagggctggaggcgtcctggcctatgagctgctgccggcactgga
cgaggtgctggctagtgacagccgcttcttgctgggcagctggctagagcaggcccgagcagcggcagtc
agtgaggccgaggccgatttctacgagcagaacagccgctaccagctgaccttgtgggggccagaaggca
acatcctggactatgccaacaagcagctggcggggttggtggccaactactacacccctcgctggcggct
tttcctggaggcgctggttgacagtgtggcccagggcatccctttccaacagcaccagtttgacaaaaat
gtcttccaactggagcaggccttcgttctcagcaagcagaggtaccccagccagccgcgaggagacactg
tggacctggccaagaagatcttcctcaaatattacccccgctgggtggccggctcttggtga
SEQ ID NO: 2:
MEAVAVAAAVGVLLLAGAGGAAGDEAREAAAVRALVARLLGPGPAADFSV
SVERALAAKPGLDTYSLGGGGAARVRVRGSTGVAAAAGLHRYLRDFCGCHVAWSG
SQLRLPRPLPAVPGELTEATPNRYRYYQNVCTQSYSFVWWDWARWEREIDWMALN
GINLALAWSGQEAIWQRVYLALGLTQAEINEFFTGPAFLAWGRMGNLHTWDGPLPP
SWHIKQLYLQHRVLDQMRSFGMTPVLPAFAGHVPEAVTRVFPQVNVTKMGSWGHF
NCSYSCSFLLAPEDPIFPIIGSLFLRELIKEFGTDHIYGADTFNEMQPPSSEPSYLAAATT
AVYEAMTAVDTEAVWLLQGWLFQHQPQFWGPAQIRAVLGAVPRGRLLVLDLFAES
QPVYTRTASFQGQPFIWCMLHNFGGNHGLFGALEAVNGGPEAARLFPNSTMVGTGM
APEGISQNEVVYSLMAELGWRKDPVPDLAAWVTSFAARRYGVSHPDAGAAWRLLL
RSVYNCSGEACRGHNRSPLVRRPSLQMNTSIWYNRSDVFEAWRLLLTSAPSLATSPA
FRYDLLDLTRQAVQELVSLYYEEARSAYLSKELASLLRAGGVLAYELLPALDEVLAS
DSRFLLGSWLEQARAAAVSEAEADFYEQNSRYQLTLWGPEGNILDYANKQLAGLVA
NYYTPRWRLFLEALVDSVAQGIPFQQHQFDKNVFQLEQAFVLSKQRYPSQPRGDTVD
LAKKIFLKYYPRWVAGSW

As used herein, the term “periphery” denotes that the vector of the invention is administrated into the circulatory system and but also in the nervous system as the vector crosses the blood brain barrier (BBB) and notably in cerebellum, spinal cord, peripheral nerves and DRG and also in the brain but more diffusely and to lesser extent than intraparenchymal delivery. According to the invention, the vector of the invention is particularly administrated intravenously.

As used herein, administration of the vector of the invention in the brain denotes an intracerebral injection (also known as intraparenchymal injection), into the white matter (periventricular white matter (frontal, parietal and occipital) notably to allow broad diffusion. In this case, the vector is delivered in the CNS.

Thus, the invention relates to a vector for use in the treatment of Sanfilippo syndrome type B (MPS3B) in a subject in need thereof, which vector comprises the full sequence of N-acetylglucosaminidase, alpha (NAGLU) encoding nucleic acid wherein the vector is administrated intravenously and intracerebrally.

As used herein, the term “gene” refers to a polynucleotide containing at least one open reading frame that is capable of encoding a particular polypeptide or protein after being transcribed or translated.

As used herein, the terms “coding sequence” or “a sequence which encodes a particular protein” or “encoding nucleic acid”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

In a preferred embodiment, the invention provides a nucleic acid construct comprising sequence SEQ ID No 1 or a variant thereof for use in the treatment of Sanfilippo syndrome type B (MPS3B).

The variants include, for instance, naturally-occurring variants due to allelic variations between individuals (e.g., polymorphisms), alternative splicing forms, etc. The term variant also includes NAGLU gene sequences from other sources or organisms. Variants are preferably substantially homologous to SEQ ID No 1, i.e., exhibit a nucleotide sequence identity of typically at least about 75%, particularly at least about 85%, more particularly at least about 90%, 91%, 02%, 93%, 94%, 95%, 96%, 97%, 98%, 99% with SEQ ID No 1. Variants of a NAGLU gene also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

As used herein, the term “subject” denotes a mammal, such as a rodent, a feline, a canine, and a primate. Particularly, a subject according to the invention is a human. Particularly, a subject according to the invention is a human with a Sanfilippo syndrome type B (MPS3B).

As used herein, the term “treatment” or “treat” refers to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness (e.g., the pattern of dosing used during therapy). A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The invention also relates to a method of treating Sanfilippo syndrome type B (MPS3B) in a subject in need thereof, said method comprising administering to the subject a therapeutically effective amount of a vector which comprises the full sequence of N-acetylglucosaminidase, alpha (NAGLU) encoding nucleic acid and wherein the vector is administrated to said subject in the venous system and directly in the brain.

As used herein, the term “vector” has its general meaning in the art and refers to the vehicle by which a nucleic acid molecule can be introduced into cells, so as to transform the cell and promote expression (e.g. transcription and/or translation) of the introduced sequence. According to the invention, vectors include viral vectors or non-viral vectors.

### Non-Viral Vectors

In a preferred embodiment, the vector use according to the invention is a non-viral vector. Typically, the non-viral vector may be a plasmid encoding NAGLU.

Particularly, a non-viral vector can be an exosome.

Non-viral vectors mainly comprise chemical systems that are not of viral origin and generally include chemical methods such as cationic liposomes and polymers. Non-viral vectors useful in the practice of the present invention has very well known in the art. According to the invention, non-viral vectors include but are not limited to liposomes such as cationic liposomes, solid-lipid nanoparticles (SLNs or LNPs) such as [(4-hydroxybutyl)azanediyl]di(hexane-6,1-diyl) bis(2-hexyldecanoate)-based nanoparticles; niosomes; polymers such as cationic polymers; polymers-based nanoparticles such polyethylenimine (PEI)-based nanoparticles; lipopeptides-based nanoparticles such as lipid 1,2-dilinoleyloxy-3-dimethylaminopropane (DLin-DMA)-based nanoparticles, dilinoleylmethyl-4-dimethylaminobutyrate (DLin-MC3-DMA)-based nanoparticles, ALC-0315-based nanoparticles, ALC-0159-based nanoparticles SM-102-based nanoparticles and; and chitosans as described in Toualbi L, et al. International Journal of Molecular Sciences, Maier. M et al. Molecular Therapy (2013), Shriane D et al. Biol Pharm Bull (2018). Non-viral vectors according to the invention include also the non-viral vectors described in patent WO2017049245 and WO2018081480.

In some embodiments, the method according to the invention, wherein the non-viral vector is cationic a polymers-based nanoparticle, and more particularly is a polyethylenimine (PEI)-based nanoparticle.

In some embodiments, the method according to the invention, wherein the vector is a non-viral vector comprising ribonucleic acid (RNA) or deoxyribonucleic acid (DNA) encoding NAGLU.

In some embodiments, the method according to the invention, wherein the vector is a non-viral vector comprising messenger ribonucleic acid (mRNA) encoding NAGLU.

### Viral Vectors

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

The terms “Gene transfer” or “gene delivery” refer to methods or systems for reliably inserting foreign DNA into host cells. Such methods can result in transient expression of non integrated transferred DNA, extrachromosomal replication and expression of transferred replicons (e.g., episomes), or integration of transferred genetic material into the genomic DNA of host cells.

Examples of viral vector include adenoviral, retroviral, lentiviral, herpesvirus and adeno-associated virus (AAV) vectors.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In a particular embodiment, adeno-associated viral (AAV) vectors are employed.

In another particular embodiment, the AAV vector is AAV1, AAV2, AAV3, AAV4, AAV5, AAV6, AAV7, AAV8, AAV9, AAVrh10 and all variants of AAV9, including AAV PHP.B (see for example the patent application WO2015038958), AAVPHP.eB, AAV-PHP.N”, and “AAV-PHP.B-DGT (see the patent application WO2017100671 or Chan Y Ken, Nat Neurosci. 2017 August; 20(8):1172-1179.), AAV3B, AAV-2i8, Rh74, AAV capB10, AAVMacPNS1 or AAVMacPNS2 or any other serotypes of AAV that can infect human, monkeys or other species.

In a more particular embodiment, the AAV vector is an AAVPHP.eB.

Thus, the invention relates to a vector for use in the treatment of Sanfilippo syndrome type B (MPS3B) in a subject in need thereof, which vector comprises the full sequence of N-acetylglucosaminidase, alpha (NAGLU) encoding nucleic acid wherein the vector is administrated to the said subject in the periphery and in the brain and wherein the vector is the AAV-PHP.eB.

By an “AAV vector” is meant a vector derived from an adeno-associated virus serotype, including without limitation, AAV-1, AAV-2, AAV-3, AAV-4, AAV-5, AAV6, AAV9 etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g, by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the CYP46A1 gene) and a transcriptional termination region.

The control elements are selected to be functional in a mammalian cell. The resulting construct which contains the operatively linked components is bounded (5′ and Y) with functional AAV ITR sequences. By “adeno-associated virus inverted terminal repeats” or “AAVITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, 1994; Berns, KI “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV6, etc. Furthermore, 5′ and 3′ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV6, etc. Furthermore, 5′ and 3′ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Particularly preferred are vectors derived from AAV serotypes having tropism for and high transduction efficiencies in cells of the mammalian CNS, particularly neurons. A review and comparison of transduction efficiencies of different serotypes is provided in Cearley C N et al., 2009. In one preferred example, AAV2 based vectors have been shown to direct long-term expression of transgenes in CNS, preferably transducing neurons. In other nonlimiting examples, preferred vectors include vectors derived from AAVrh10 serotype, which have also been shown to transduce cells of the CNS and particularly deep grey nucleus and diffusely cortex and white matter.

The selected nucleotide sequence is operably linked to control elements that direct the transcription or expression thereof in the subject in vivo. Such control elements can comprise control sequences normally associated with the selected gene.

Alternatively, heterologous control sequences can be employed. Useful heterologous control sequences generally include those derived from sequences encoding mammalian or viral genes. Examples include, but are not limited to, the phophoglycerate kinase (PKG) promoter, CAG, neuronal promoters, promoter of Dopamine-1 receptor and Dopamine-2 receptor, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, CA). For purposes of the present invention, both heterologous promoters and other control elements, such as CNS-specific and inducible promoters, enhancers and the like, will be of particular use.

Examples of heterologous promoters include the CMV promoter. Examples of CNS specific promoters include those isolated from the genes from myelin basic protein (MBP), glial fibrillary acid protein (GFAP), and neuron specific enolase (NSE).

Examples of inducible promoters include DNA responsive elements for ecdysone, tetracycline, hypoxia andaufin.

The AAV expression vector which harbors the DNA molecule of interest bounded by AAV ITRs, can be constructed by directly inserting the selected sequence (s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publications Nos. WO 92/01070 (published 23 Jan. 1992) and WO 93/03769 (published 4 Mar. 1993); Lebkowski et al., 1988; Vincent et al., 1990; Carter, 1992; Muzyczka, 1992; Kotin, 1994; Shelling and Smith, 1994; and Zhou et al., 1994. Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS cells (notably neurons) can be used. The complete chimeric sequence is assembled from overlapping oligonucleotides prepared by standard methods. See, e.g., Edge, 1981; Nambair et al., 1984; Jay et al., 1984. In order to produce rAAV virions, an AAV expression vector is introduced into a suitable host cell using known techniques, such as by transfection. A number of transfection techniques are generally known in the art. See, e.g., Graham et al., 1973; Sambrook et al. (1989) Molecular Cloning, a laboratory manual, Cold Spring Harbor Laboratories, New York, Davis et al. (1986) Basic Methods in Molecular Biology, Elsevier, and Chu et al., 1981. Particularly suitable transfection methods include calcium phosphate co-precipitation (Graham et al., 1973), direct microinjection into cultured cells (Capecchi, 1980), electroporation (Shigekawa et al., 1988), liposome mediated gene transfer (Mannino et al., 1988), lipid-mediated transduction (Felgner et al., 1987), and nucleic acid delivery using high-velocity microprojectiles (Klein et al., 1987).

For instance, a preferred viral vector, such as the AAVrh10, comprises, in addition to a cholesterol 24-hydroxylase encoding nucleic acid sequence, the backbone of AAV vector with ITR derived from AAV-2, the promoter, such as the mouse PGK (phosphoglycerate kinase) gene or the cytomegalovirus/0-actin hybrid promoter (CAG) consisting of the enhancer from the cytomegalovirus immediate gene, the promoter, splice donor and intron from the chicken β-actin gene, the splice acceptor from rabbit β-globin, or any neuronal promoter such as the promoter of Dopamine-1 receptor or Dopamine-2 receptor with or without the wild-type or mutant form of woodchuck hepatitis virus post-transcriptional regulatory element (WPRE).

### Delivery of the Vectors

It is herein provided a method for treating Sanfilippo syndrome type B (MPS3B) in a subject in need thereof, said method comprising:
(a) providing a vector as defined above, which comprises a NAGLU encoding nucleic acid; and
(b) delivering the vector in the periphery and in the brain.

According to the invention, the vector can be administrated intravenously and in the brain simultaneous, separately or sequentially.

As used herein, the term “simultaneous use” denotes that the intravenously and the intracerebrally administration occur at the same time.

As used herein, the term “separate use” denotes that the intravenously and the intracerebrally administration does not occur at the same time.

As used herein, the term “sequential use” denotes that the intravenously and the intracerebrally administration occur by following an order.

To deliver the vector specifically to a particular region and to a particular population of cells, the vector may be administered by intracerebral injection. For example, patients have the stereotactic frame base fixed in place (screwed into the skull). The brain with stereotactic frame base (MRI compatible with fiducial markings) is imaged using high resolution MRI. The MRI images are then transferred to a computer which runs stereotactic software. A series of coronal, sagittal and axial images are used to determine the target (site of AAV vector injection) and trajectory. The software directly translates the trajectory into 3 dimensional coordinates appropriate for the stereotactic frame. Alternatively, neuronavigation can be used instead of the stereotactic procedure, depending on neurosurgeon preference. Burr holes are drilled above the entry site and the stereotactic apparatus positioned with the needle implanted at the given depth. The AAV vector is then injected at the target sites. Since the AAV vector integrates into the target cells, rather than producing viral particles, the subsequent spread of the vector is minor, and mainly a function of passive diffusion from the site of injection and of course the desired transsynaptic transport, prior to integration. The degree of diffusion may be controlled by adjusting the ratio of vector to fluid carrier.

Additional routes of administration may also comprise local application of the vector under direct visualization, e.g., superficial cortical application, or other nonstereotactic application.

However the invention encompasses delivering the vector to biological models of the disease. In that case, the biological model may be any mammal at any stage of development at the time of delivery, e.g., embryonic, fetal, infantile, juvenile or adult, preferably it is an adult.

Particularly, the method of the invention comprises intracerebral administration through stereotaxic or neuronavigated injections. However, other known delivery methods may also be adapted in accordance with the invention. For example, for a more widespread distribution of the vector across the CNS (for an intracerebral injection in the periventricular white matter), it may be injected into the cerebrospinal fluid, e.g., by lumbar puncture. To direct the vector to the peripheral nervous system, it may be injected into the spinal cord or into the peripheral ganglia, or the flesh (subcutaneously or intramuscularly) of the body part of interest. In certain situations the vector can be administered via an intravascular approach. For example, the vector can be administered intra-arterially (carotid) in situations where the blood-brain barrier is disturbed or not disturbed. Moreover, for more global delivery, the vector can be administered during the “opening” of the blood-brain barrier achieved by infusion of hypertonic solutions including mannitol or by any mechanical action.

The vectors used herein may be formulated in any suitable vehicle for delivery. For instance they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

The preferred doses and regimen may be determined by a physician, and depend on the age, sex, weight, of the subject, and the stage of the disease. As an example, for delivery of NAGLU using a viral expression vector, each unit dosage of NAGLU expressing vector may comprise 2.5 to 10 μl of a composition including a viral expression vector in a pharmaceutically acceptable fluid at a concentration ranging from 10⁹ to 10¹⁷ viral genome per ml for example.

### Pharmaceutical Composition

A second object of the invention concerns a pharmaceutical composition for use in the treatment of Sanfilippo syndrome type B (MPS3B) in a subject in need thereof which comprises a therapeutically effective amount of a vector according to the invention.

By a “therapeutically effective amount” is meant a sufficient amount of the vector of the invention to treat Sanfilippo syndrome type B at a reasonable benefit/risk ratio applicable to any medical treatment.

It will be understood that the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range per adult per day. The therapeutically effective amount of the vector according to the invention that should be administered, as well as the dosage for the treatment of a pathological condition with the number of viral or non-viral particles and/or pharmaceutical compositions of the invention, will depend on numerous factors, including the age and condition of the patient, the severity of the disturbance or disorder, the method and frequency of administration and the particular peptide to be used.

The presentation of the pharmaceutical compositions that contain the vector according to the invention may be in any form that is suitable for intraparenchymal, intracisternal, intrathecal, intraventricular or intravenous administration.

In the pharmaceutical compositions of the present invention for intravenous, intrathecal, intraventicular, intraparenchymal or intracisternal administration, the active principle, alone or in combination with another active principle, can be administered in a unit administration form, as a mixture with conventional pharmaceutical supports, to animals and human beings.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The vector according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

Multiple doses can also be administered.

The invention will be further illustrated by the following figures and examples. However, these examples and figures should not be interpreted in any way as limiting the scope of the present invention.

### FIGURES

FIG. 1: (A) NaGlu activity in the brain, NaGlu activity was measured in tissue homogenates prepared from the mice brain hemispheres; (B) GAG accumulation in the brain, GAG concentration was determined in mice cortical tissue extracts; (C) mRNA level of MIP1α in brain tissue, mRNA amounts are expressed relative to the reference ARPO mRNA; (D) Behavior analysis, mice were tested for behavior at 22 weeks post-injection in an elevated plus maze test. Data are means±SEM. *p<0.05; **p<0.01; ***p<0.001 were considered significant.

FIG. 2: NaGlu activity in the brain, NaGlu activity was measured in tissue homogenates prepared from the mice brain hemispheres.

FIG. 3: MRI T2-weighted images for each 3 NHP, at immediate post op scan, 1 w post-surgery and 6 weeks post-surgery. In all NHP, T2 hyperintense signals were detected at least on one out of 6 injection sites at 6 weeks. (NHP1: 1 out of 3 on AAV side and 0 out of 3 on vehicle side; NHP2, 1 out of 6 site of injection and NHP3: 2 out of 6 sites of injections.

FIG. 4: NAGLU activity in the brain of NHP(A), in other CNS and PNS structures (B) and in peripheral tissues (C) and serum (D). For A: black correspond to IV only in NHP1, and white of the mean values of NHP1 to 3 of IV+IC, for B-C: mean values of NHP1 to 3 of IV+IC and for C individual data in serum * white matter for IV only was not available. Dotted line corresponds to mean basal NAGLU activity in non-injected NHP.

FIG. 5: Haematology and blood parameters of control and MPS3B treated dog with AAVPHP.eB IV+IC.

FIG. 6: MRI T2 and T1-weighted images for MPS3B treated dog, at immediate post op scan, 1 w post-surgery and 12 weeks post-surgery. Immediate post op imaging demonstrates a proper targeting of the white matter. At 1 week post op, MRI parameters is fully normal without even seeing the injection site. At 3 months post op, signal of local inflammation at the entry point; that correlate with macroscopic observation on the brain tissue, most probably due to local immune response against the Naglu enzyme.

FIG. 7: NAGLU activity in the brain of MPS3B treated dog and other CNS and PNS regions as well as spleen, and liver. Activity is expressed in percent compare to the WT control dog.

FIG. 8: NAGLU activity in CSF of MPS3B treated dog Activity is expressed in nmol/h/mL of CSF and compare to the WT control dog.

FIG. 9: Glycosaminoglycans (GAG) measurement in brain, CNS, PNS and peripheral tissues of MPS3B treated dog and control dog.

### EXAMPLES

### Example 1: Mouse and NHP Study

### Material and Methods:

### MPSIIIB Mice Experiments

In our study, we used a mouse model of MPSIIIB created by Li and his colleagues (Li et al., 1999). Knock-out Animals (with no expression of N-acetylglucosaminidase enzyme) show an accumulation of heparan sulfate glycosaminoglycan (HS GAG) in tissues, an elevation of GM2 and GM3 gangliosides, a widespread activation of microglia (Ohmi et al., 2003), and vacuolation in brain cells at early stages of the disease. Abnormal behavior was reported (Li et al., 1999).

Proof of concept of this approach has been established in the mouse model of MPSIIIB and due to the high efficiency of AAVPHP.eB delivery based on previous results, only intravenous delivery was performed, and combination approach maintained only for large animals as in Humans. AAVPHP.eB-NaGlu was injected at a total dose of 5E11vg in the tail vein of 4 weeks old MPSIIIB mice and their age-matched wild type (WT) littermates and necropsied at 4-, 12-, and 22-weeks post-injection.

### N-acetylglucosaminidase (NaGlu Assays)

Deeply anesthetized animals were perfused with PBS. Lysosomal enzyme activity levels were equivalent in both conditions when assayed in control mice. About 30 mg of tissue was homogenized by hand in 500 μl of 0.9% NaCl and 0.2% Triton X-100, rotated for 2 hr at 4° C., and centrifuged to remove debris. The activity of a-Nacetylglucosaminidase was determined fluorometrically. Homogenate (25 μll) was incubated for 17 hr at 37° C. with an equal volume of 0.2 mM 4-methylumbelliferyl a-N-acetylglucosaminide in 0.1M sodium acetate buffer, pH 4.3, containing 0.5 mg/ml BSA; the fluorescence of 4-methylumbelliferone released was measured after addition of 1.0 ml of glycine buffer, pH 10.5.

### Analysis of Glycosaminoglycans (GAG) in Brain Extracts.

Frozen samples were homogenized with a minimum volume of water (10% vol/weight). Defatted pellets were dried and weighed. Dried residues were digested overnight at 65 uC with papain (0.3% w:v) in 3 ml of 100 mM sodium acetate buffer pH 5.5 containing 5 mM cysteine and 5 mM EDTA. After centrifugation, GAGs were measured in the supernatant with a dimethylmethylene blue dye binding assay. Briefly, 200 ml of the supernatant was added to 2.5 ml of dimethylmethylene blue reagent and the absorbance at 535 nm was measured. Heparan sulfate was used as standard. Data (means of duplicates) were expressed as mg of GAGs per mg of dried pellet.

### Quantitative RT-PCR

Total RNA was extracted using SV Total RNA Isolation System from 30 mg of cortex and suspended in 50 μl of water. One microgram of total RNA was used to synthesize cDNA. Quantitative PCR was performed in a LightCycler 480 (Roche) with the SYBR Green PCR Master Mix (Roche). Ct (Cycle threshold) values were determined as the numbers of PCR cycles at which specific amplification of the target sequence occurred. Ct superior to 38 were considered as background signal. cDNA amounts were expressed as 2exp(Ct1−Ct2), in which Ct1 is a reference Ct measured for the amplification of ARPO (Acidic ribosomal phosphoprotein) cDNAs and Ct2 is the Ct measured for the amplification of the examined cDNA. Samples were analyzed in triplicate.

### Elevated Plus Maze Test

We used a cross-maze built with opaque Plexiglas panels consisting of two opposing open arms (length, 30.5 cm; width, 7 cm) and two walled arms (same size with 18-cm-high walls) extending a central square platform. The maze is 54 cm above floor level. Testing was performed under red-light illumination (dark condition) with intensity set up to 1 lux. Animals entered the maze on the central platform, facing an open arm. Each trial (10 min) was videotaped for off-line analyses. Time spent in arms was scored when animals put two forepaws within an arm and continued until they exited the arm. To reduce activity-induced artifacts, data were expressed as ratio of time spent in open arms relative to total time spent in arms during a 10 min session.

### NHP Experiments:

### Adeno-Associated Viral Vector Construction and Production

AAV vectors were produced and purified by Atlantic Gene therapies (Translational Vector Core Research grade services, Nantes, France). AAVPHP.eB-CAG Naglu was produced by cloning the NAGLU sequence under the CAG promoter. The viral constructs for pAAVPHP.eB-CAG-hNAGLU contained the expression cassette consisting of the human NAGLU genes, driven by a CMV early enhancer/chicken b-actin (CAG) synthetic promoter surrounded by inverted terminal repeats (ITR) sequences of AAV2. Plasmid for AAVPHP.eB was obtained from Addgene (United States). The final titer of the batch was 4.1×1013 vector genomes (vg)/ml.

### Animal Model

All animal studies were performed in accordance with local and national regulations and were reviewed and approved by the relevant institutional animal care and use committee. NHP were housed in a temperature- and humidity-controlled animal facility (target temperature 20-24° C., no specific humidity target in the guidelines (20 to 60%) with a 12 h light-dark cycle (no record for light). NHP diet pellets (ref 307, Safe) were given ad libitum except during the fasting experimental period and each day animal received one fruit or one vegetable and seeds or dry fruits. Tap water will be offered ad libitum in polycarbonate bottles.

### Injection of AAV Vector

Three female monkeys received 1×1013vg total dose of AAVPHP.eB-CAG-NAGLU in the saphenous vein and unilateral for NHP1 or bilatrela delivery of AAVPHP.eB-NAGLU in 3 locations par hemisphere with 1E11vg per deposit in 60 uL. Then, the monkeys were treated with corticoids for 8 days (from D−1 to D7) following injection. The monkeys were followed-up during 6 weeks, with blood sampling at 1, 3 and 6 weeks. NAGLU activity was measured in the cerebrospinal fluid, serum and in various CNS and peripheral tissues.

A control animal that received AAVPHP.eB at the same concentration but with another transgene not related to NAGLU was used as a negative control for NAGLU activity to evaluate background expression.

TABLE 1
protocol of AAV injections

| | | | Intravenous | Intraparenchymal dose of | |
|---|---|---|---|---|---|
| | | | dose of | AAVPHP.eB-CAG-NaGlu | |
| | | | AAVPHP.eB- | Left | Right |
| | Animal | Name and | CAG-NaGlu | hemisphere | hemisphere |
| Nb | nb | Tatoo | (vg) | (vg/uL) | (vg/uL) |
| NHP | 1 female | PINK | 1E¹³ | Vehicle | 1.65E⁹ vg/uL |
| 1 | | MF1810 | | | and 60 uL per |
| | | | | | deposit = |
| | | | | | 1E11vg |
| NHP | 1 female | BLUE | 1E¹³ | 1.65E9 vg/uL | 1.65E⁹ vg/uL |
| 2 | | CIB045 | | and 60 uL per | and 60 uL per |
| | | | | deposit = | deposit = |
| | | | | 1E11vg | 1E11vg |
| NHP | 1 female | PURPLE | 1E¹³ | 1.65E9 vg/uL | 1.65E⁹ vg/uL |
| 3 | | CIB051 | | and 60 uL per | and 60 uL per |
| | | | | deposit = | deposit = |
| | | | | 1E11vg | 1E11vg |

### Tissue Preparation

Animals were sacrificed by an intravenous administration of a lethal dose of Euthasol (140 mg/kg, Vetcare) 6 weeks after treatment. NHP was perfused intracardiacally with phosphate buffered saline (PBS), followed by 500 mL of PFA 4%. Brain, spinal cord, sciatic nerve, heart, liver, gall gladder, lung, spleen and kidney were collected for analysis. Different structures of a cerebral hemisphere (frontal, temporal and occipital cortex, caudate, putamen, hippocampus, thalamus, corpus callosum, cerebellar white matter, pons) were dissected and frozen in liquid nitrogen. Spinal cord, sciatic nerve, dorsal root ganglia (DRG) heart, liver, lung, spleen and kidney were directly frozen in liquid nitrogen and stored in −80° C. For protein extraction from the same samples, tissue samples were crushed in liquid nitrogen and divided into two equals parts.

The brain was divided in 2 and one hemisphere was devoted to histological analysis and cryopreserved as well as a portion of spinal cord, sciatic nerve were post-fixed overnight in 4% paraformaldehyde (PFA)/PBS1×. Brain samples were rinsed three times in PBS 1× and or frozen after PBS-sucrose gradients (5%, 10% and finally 20%) for 3-4 days each and cut into 40-um coronal section of brain. All other organs were embedded in paraffin and cutted for transversal section of spinal cord or 5 um coronal section for the brain.

### Protein Extraction and NAGLU Activity Quantification

Samples were homogenized in lysis buffer and NAGLU activity done as described previously.

### Results:

### Mouse In Vivo Study

### Simple Injection

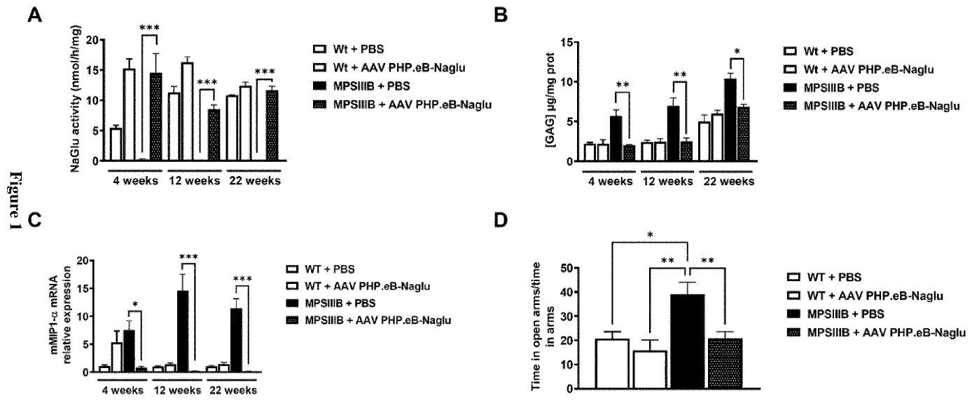

Vector genomes were detected in the brain (data not shown) and high NaGlu activity was measured in the brain starting 4 weeks post-injection and persisted after 22 weeks post-injection (FIG. 1A). Interestingly, the values obtained are superior to those obtained after treatment with AAV9. High NaGlu activity was also measured in cerebellum, spinal cord, and liver (data not shown). This was accompanied by the normalization of GAG storage levels (FIG. 1i). MIP1α mRNA was dramatically increased in MPSIIIB mice starting the age of 8 weeks (FIG. 1C). Delivery of the missing enzyme NaGlu in the brain of MPSIIIB mice normalized MIP1α mRNA level starting 4 weeks post-treatment (FIG. 1C). TNFα, IL-1β mRNAs were also decreased after treatment (data not shown). MPSIIIB mice were scored in the elevated plus maze, rotarod test, and object recognition test at 22 weeks post-injection. In the open field test, time devoted in open arms significantly decreased in MPSIIIB treated mice and did not differ from wild-type mice (FIG. 1D). These results showed that behavioral scores assessing anxiety were improved in MPSIIIB mice after a single injection of AAVPHP.eB-NaGlu. Motor coordination and balance were also assessed by the rotarod test, treated MPSIIIB mice spent more time on the apparatus before falling (data not shown). MPSIIIB treated mice showed also an amelioration in the memory functions after novel object recognition test (data not shown). Our data indicate that a single IV injection of AAVPHP.eB-NaGlu vector crosses the blood-brain barrier and allowed widespread NaGlu delivery in brains of MPSIIIB mice. In addition, NaGlu delivery in the CNS was followed by a correction of lysosomal storage pathology, a reduction of established neuroinflammation, and an improvement in motor and cognitive functions.

### Double Injection

In order to study the effect of the vector in double injection, IV injection at 5E11vg was combined to an intraparenchymal injection of AAVPHP.eB-NaGlu at 1E9vg 4 weeks old MPSIIIB mice. High NaGlu activity was measured in the brain starting 4 weeks post-injection in both treated group in comparison to untreated MPSIIIB mice. A significant increase of NaGlu activity was observed in mice brain with double injection.

### NHP Experiments:

### Preclinical Study in Non-Human Primates (NHP)

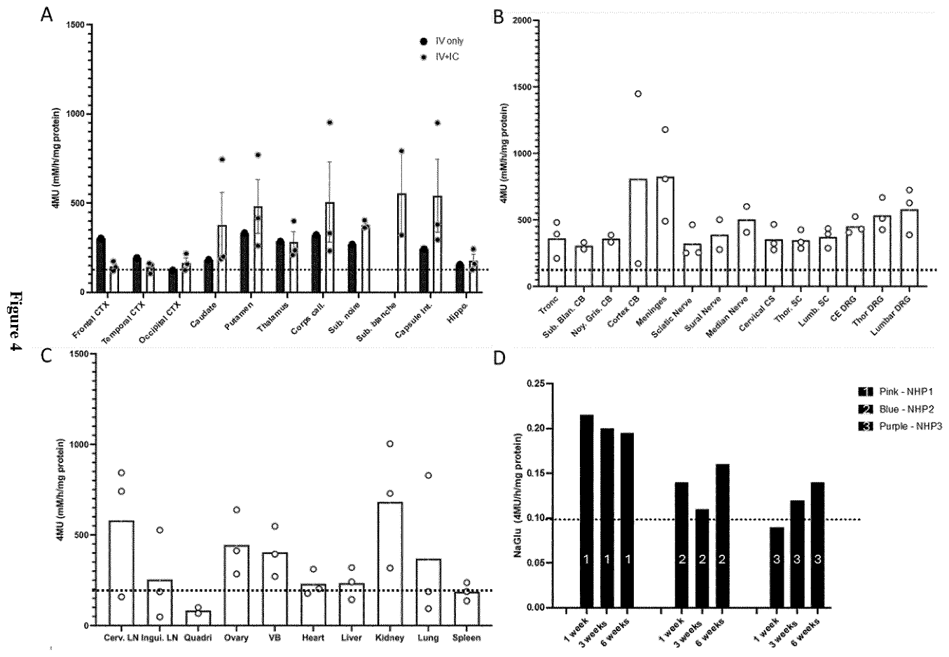

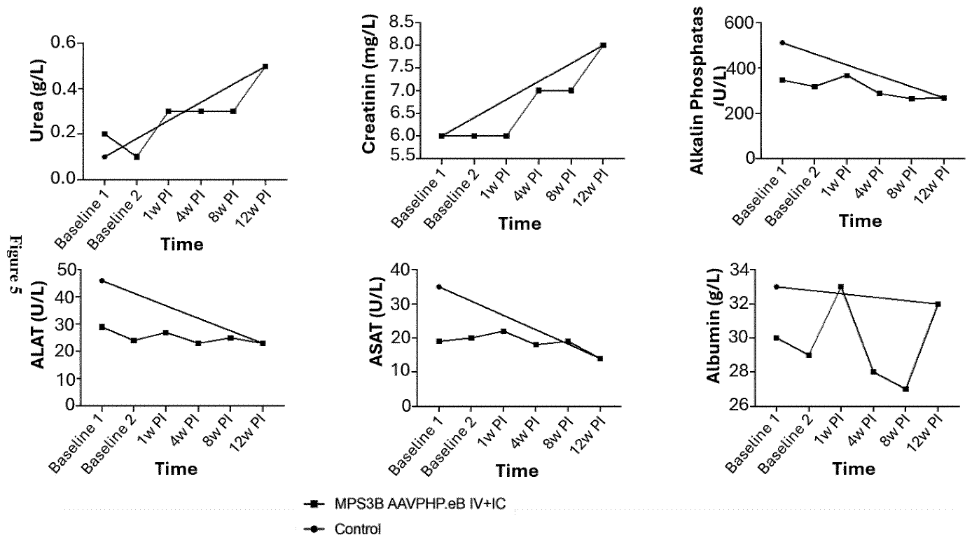

The purpose of this NHP study was to perform a biodistribution, efficiency and first assessment of tolerance study in NHP combining 6 intraparenchymal deliveries (1E¹¹vg each deposit) in the periventricular white matter (frontal, parietal and occipital) to maximize AAV spreading and intravenous delivery of AAVPHP.eB-CAG-NaGlu at 1E¹³ total dose with 4 animals. Intraparenchymal delivery was done using first generation Cornell catheters guided by neuronavigation as further described. The Medtronic neuronavigation system that allows accurate targeting of the white matter, as well as Convection-Enhanced Delivery (CED) was used simultaneously with each catheter. After dosing, animals were followed up for 6 weeks. All the study was run as a GLP-like study at the TIDU-GENOV with appropriate quality follow up. All NHP were followed for 6 weeks following surgery prior to necropsies. Combine IV and intracerebral approach was perfectly well tolerated in all animals, notably with no body weight change and normal blood formulation and ionogram all along the study. Importantly, NHP were monitored daily during the in-life period and none of them present any neurological symptoms or abnormalities, they all perfectly well tolerated the administration procedure. All NHP undergo MRI follow up with immediate post op MRI, 1 w and 6 w prior to necropsy time point with each time T2-weighted and T2* weighted sequences. Immediately post-surgery, we observed almost always hyperintense signal at least at one site of injection, at 1 week PI, all MRI were perfectly normal. At 6 weeks post injection, some T2w hyperintense signals were observed in all NHPs at least at 1 injection site and in one animal a mild uptake of gadolinium was observed suggesting an opening of the BBB limited at the injection site that could remain form the surgical procedure but that seems not associated with any active inflammation (FIG. 3).

To complete tolerance study, evaluation of a well know biomarker for neuronal integrity: NF-L is ongoing both in serum and CSF. Finally, evaluation of immune response is ongoing is serum for humoral immunity, with evaluation of neutralizing antibodies against both AAVPHP.eB and NAGLU as well as investigation of cellular immunity with IFN-ELISPOT on PBMCs for both AAV and transgene.

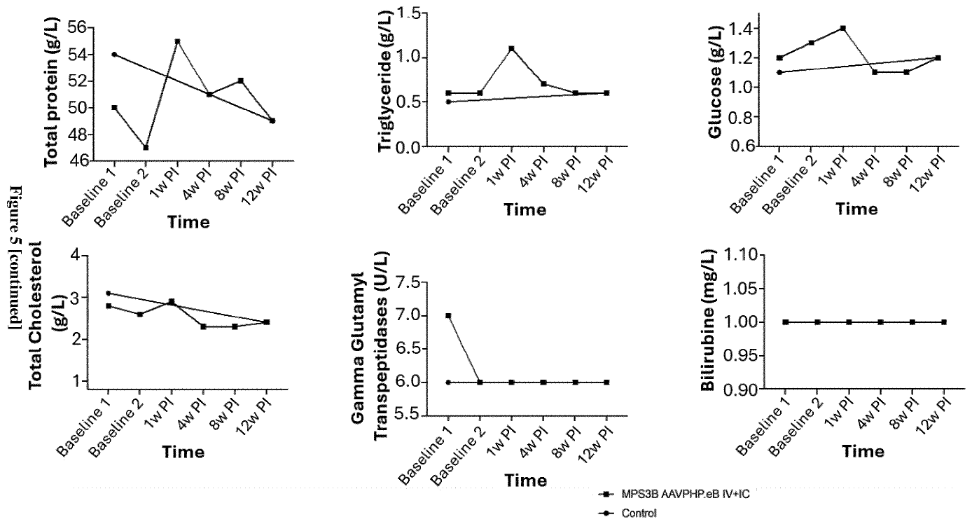

The second crucial point of our study was the evaluation of NAGLU activity in the brain and more largely in the whole CNS, the peripheral organs as in the sera of injected animals (FIG. 4).

A significant NAGLU activity was detected in brain regions, with a clear difference of IV+IC delivery demonstrating a significant increase in NAGLU expression notably in white matter (corpus callosum, general white matter, internal capsule) but also in caudate and putamen (FIG. 4).

For all other regions, no difference was observed between IV only and IV+IC delivery (FIG. 4). In addition, NAGLU activity was quantified in all CNS and PNS tissues and significant activity was detected in all tissues with higher detected activity in the cerebellum and meninges. In peripheral organs, NAGLU activity was mostly in the range of non-injected controls.

Finally, in serum, significant increase of NAGLU activity was detected in all three animals compare to baseline.

Finally, regarding tolerance of the approach, we first evaluated overall tolerance with H&E staining of all brain and CNS sections as well as peripheral organs. Regarding the brain, perivascular cuffings were observed in both Vehicle and AAV injected hemispheres, in vehicle treated hemisphere, there are restricted on the needle track and with low intensity. In AAV injected hemisphere, perivascular was a bit more present with a mild score but restricted to the injection site. These findings was well documented in previous NHP study for several AAV serotypes (AAV2, 5 and rh10) and results from local reaction to the AAV (Piguet colle 2010 and piguet zerah 2015).

### Example 2: Canine In Vivo Study

### Material and Methods

### Adeno-Associated Viral Vector Construction and Production

AAV vectors were produced and purified by Atlantic Gene therapies (Translational Vector Core Research grade services, Nantes, France). AAVPHP.eB-CAG Naglu was produced by cloning the NAGLU sequence under the CAG promoter. The viral constructs for pAAVPHP.eB-CAG-hNAGLU contained the expression cassette consisting of the human NAGLU genes, driven by a CMV early enhancer/chicken b-actin (CAG) synthetic promoter surrounded by inverted terminal repeats (ITR) sequences of AAV2. Plasmid for AAVPHP.eB was obtained from Addgene (United States). The final titer of the batch was 4.1×1013 vector genomes (vg)/ml.

### Animal Model

The animal model used correspond to the canine model of MPS3B pathology as described elsewhere. In accordance with French and European regulations for the protection of animals used for experimental and other scientific purposes (Directive 2010/63/EU of the European Parliament and of the Council of 22 Sep. 2010 on the protection of animals used for scientific purposes and the National Transposition, 1 Feb. 2013), the Center is subjected to regular inspection from Direction Départementale de la Protection des Populations, Veterinary & Environment Services inspections to ensure that the staff is operating to the required animal welfare standards and is complying with the conditions of their authorization(s) and the requirements of the scientific animal protection legislation.

All animals will be generated at the facility from heterozygous breedings.
Caging: The animals will be housed in pair. During the study, the animals will be housed according to the HCB biosafety requirements.
Housing: one room for the study, in an air-conditioned building:
   temperature: 18-21° C. (target range),
   relative humidity: 40-70% (target range),
   air changes: approximatively 10 to 12 changes per hour—light cycles: 12 hours light (artificial)/12 hours dark.
Environmental enrichment: structural enrichment in cages (shelves), toys (Balls, Kongs®, . . . ), socialization dogs/Human, music program.
Diet: Kibbles adapted to the species, weight and age of the animal will be distributed once a day.
Water: human consumption quality water will be given ad libitum.

### Dog Genotyping

Swab samples will be taken between 7 and 15 days of age of the puppies and were shipped at RT to the study director for DNA extraction and IDUA and NAGLU genotyping according to internal protocol.

### AAV Delivery

One male dog received 1×1013vg total dose of AAVPHP.eB-CAG-NAGLU in the saphenous vein and bilateral delivery of AAVPHP.eB-NAGLU in 2 locations par hemisphere with 1E11vg per deposit in 60 uL using Medtronic Stel Health Station S8.

Then, the dog was treated with corticoids for 8 days (from D−1 to D7) following injection and antibiotics. Due to the absence of NagLu protein in the model we also place the dog under immunosuppressive therapy.

Animals will be pre-dosed with immunosuppressive treatment prior test item injection.

Immunosuppressive administration will continue until the end of the study. The precise immunosuppressive protocol is as follows:
Temporary treatment of cortisone:
   D−1 and D+1: 3 mg/kg/day per os of cortisone (Oromedrol®)
   D0: 1 mg on IV of cortisone (Solumedrol®)
   D+2 to D+8: 1 mg/kg/day per os of cortisone (Oromedrol®)
Treatment throughout the study:
   D−15 to Deutha: oral immunosuppressive drug (Cellcept) based on the weight with 2 treatment per day during week and only once a day during weekend. Reassessment of dosage once a week.

The dog was dosed at 3 month and followed-up during 3 months, with blood sampling and CSF at 1 week, 1.5 months and 3 months. A control dog was also necropsied to serve as a control.

NAGLU activity was measured in the cerebrospinal fluid, serum and in various CNS and peripheral tissues.

### MRI

Before performing MRI on the animals to be injected, one MRI will be carried out on the 2 non-injected animals, at 3 months of age and, in order to define and optimize image processing conditions. To compare pathology signs on MRI, a second MRI will be performed on the same dogs at 6 months of age for one and 12 months of age for the other, corresponding to the age of the injected animals.

T1w and T2w images will be performed prior to the surgery (at least 1 week before surgery) on a 1.5 T Siemens Apparatus.

T2w and T2w* sequences will be performed on DO immediately after surgery, D7±1, and 3 months post-surgery for the cohort short term (M3±3) MRI will be carried out the day before euthanasia.

The MRI will be done with a compatible frame (on loan from ICM) and a vitamin D or any other marker to lateralize images.

All sequences will be performed using gadolinium injection.

For preoperative MRI only, the animal will be placed in a stereotaxic frame in the sphinx position, with its head secured by ear bars and supported with a mouth bar. A local anesthetic gel (lidocaine) will be applied to the ear bars to treat any pain+eye gel.

MRI post-injection will be performed in the lateral decubitus position.

### Tissue Preparation

Animals were sacrificed by an intravenous administration of a lethal dose of Euthasol (140 mg/kg, Vetcare) 6 weeks after treatment. NHP was perfused intracardiacally with phosphate buffered saline (PBS), followed by 500 mL of PFA 4%. Brain, spinal cord, sciatic nerve, heart, liver, gall gladder, lung, spleen and kidney were collected for analysis. Different structures of a cerebral hemisphere (frontal, temporal and occipital cortex, caudate, putamen, hippocampus, thalamus, corpus callosum, cerebellar white matter, pons) were dissected and frozen in liquid nitrogen. Spinal cord, sciatic nerve, dorsal root ganglia (DRG) heart, liver, lung, spleen and kidney were directly frozen in liquid nitrogen and stored in −80° C. For protein extraction from the same samples, tissue samples were crushed in liquid nitrogen and divided into two equals parts. The brain was divided in 2 and one hemisphere was devoted to histological analysis and cryopreserved as well as a portion of spinal cord, sciatic nerve were post-fixed overnight in 4% paraformaldehyde (PFA)/PBS1×. Histological samples were rinsed three times in PBS 1× and were embedded in paraffin and cutted for transversal section of spinal cord or 5 um coronal section for the brain.

### Protein Extraction and NAGLU Activity Quantification

Samples were homogenized in lysis buffer and NAGLU activity done as described previously.

### Results

The purpose of this dog study was to perform a tolerance and efficacy study in the dog model of the pathology by evaluating biodistribution, efficacy, and first assessment of tolerance study in 3-month-old MPS3B dogs combining intraparenchymal delivery in the white matter and intravenous delivery of AAVPHP.eB-CAG-NaGlu. Intraparenchymal delivery were done using Cornell catheters guided by neuronavigation.

The Medtronic neuronavigation system that allows accurate targeting of the white matter, as well as Convection-Enhanced Delivery (CED) was used simultaneously with each catheter.

After dosing, animals was followed up for 3 months post-dosing to determine behavioral improvement, biodistribution, efficacy, and tolerance of AAVPHP.eB-CAG-NaGlu.

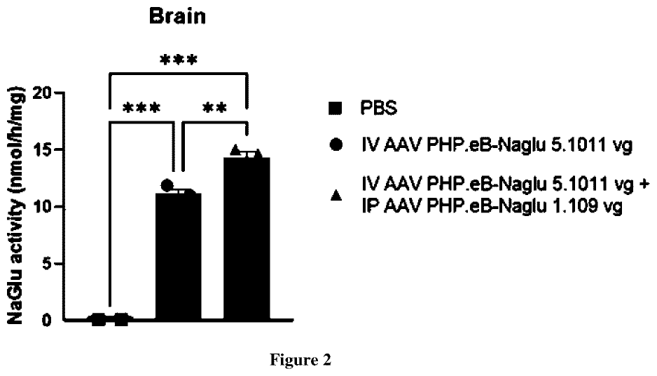

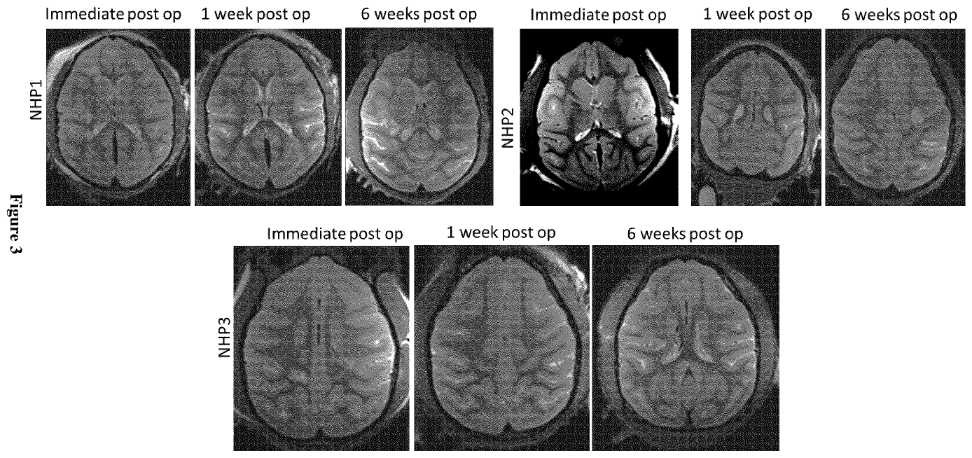

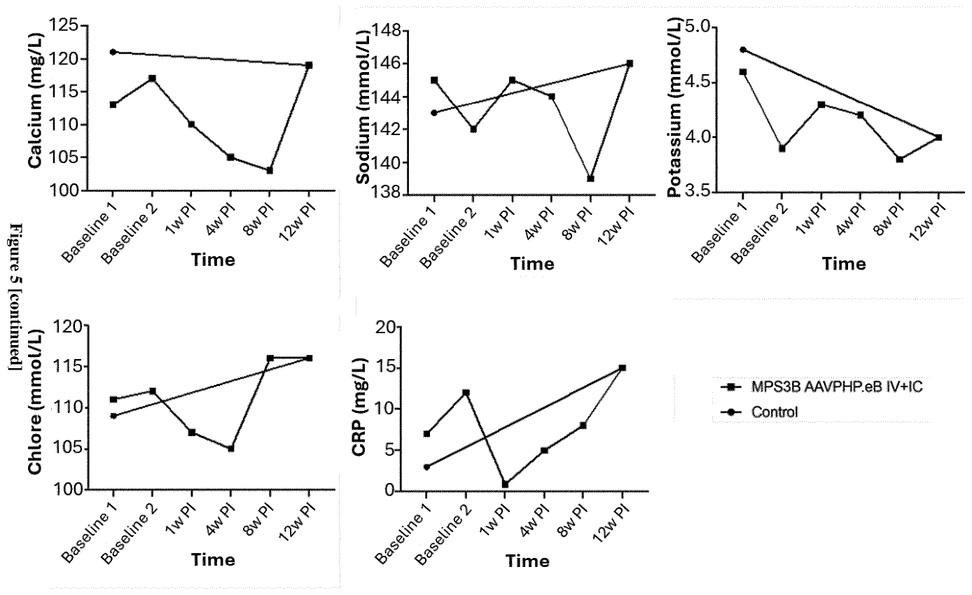

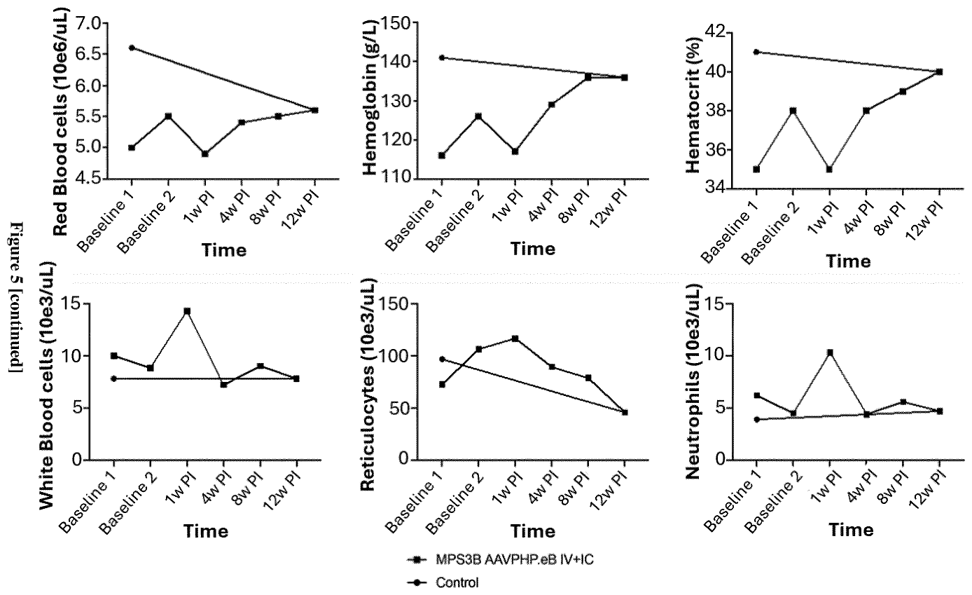

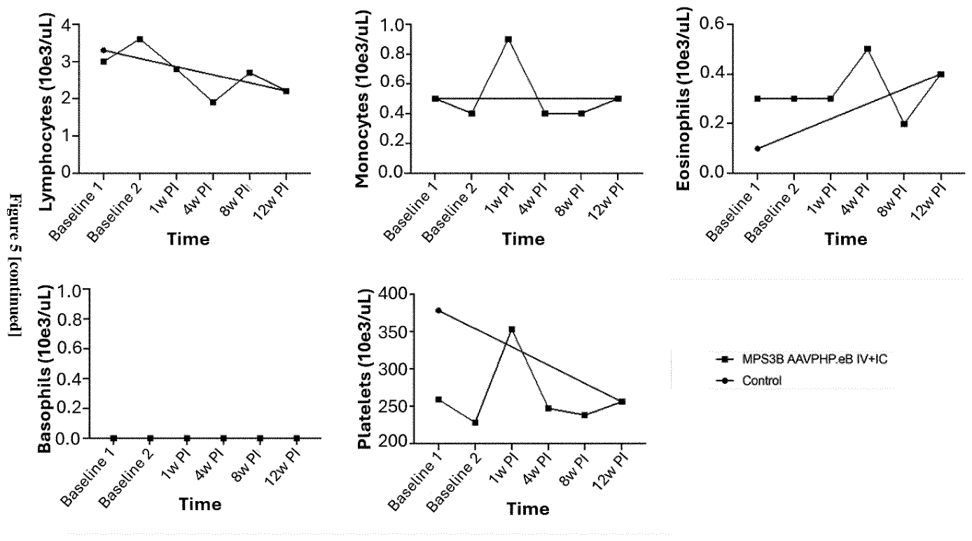

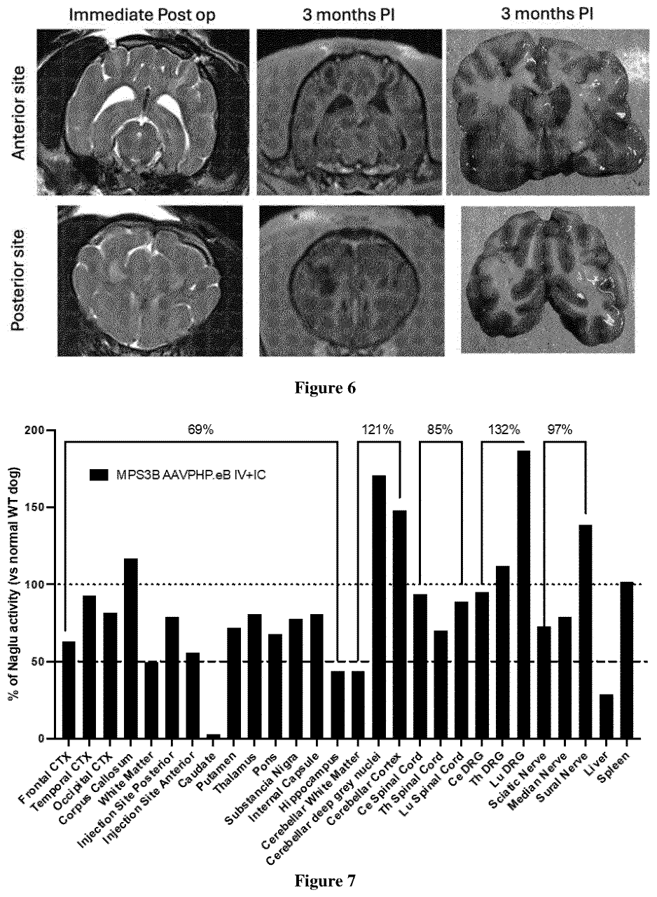

Combine IV and intracerebral approach was perfectly well tolerated in treated dog, notably with no body weight change and normal blood formulation and ionogram all along the study (FIG. 5). Importantly, dogs were monitored daily during the in-life period and none of them present any neurological symptoms or abnormalities else than their pathology (neurological signs at exams present before AAV delivery), they all perfectly well tolerated the administration procedure. Treated Dog undergo MRI follow up with immediate post op MRI, 1w and 12w prior to necropsy time point with each time T2-weighted and T2* weighted sequences with Gadolinium injection. Immediately post-surgery, we observed hyperintense signal at all delivery sites (FIG. 2), at 1w MRI did not display any abnormalities. At 3 months post op, signal of local inflammation at the entry point; that correlate with macroscopic observation on the brain tissue, most probably due to local immune response against the Naglu enzyme (FIG. 6).

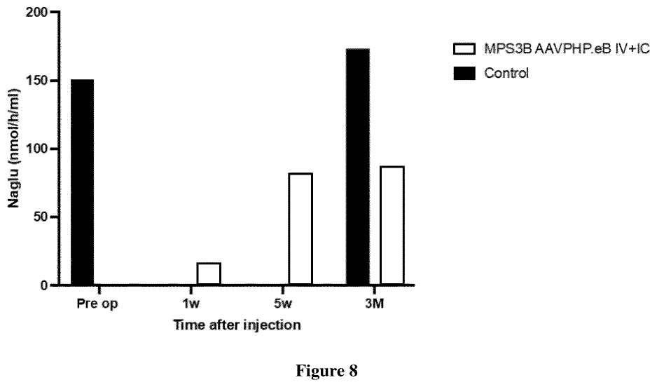

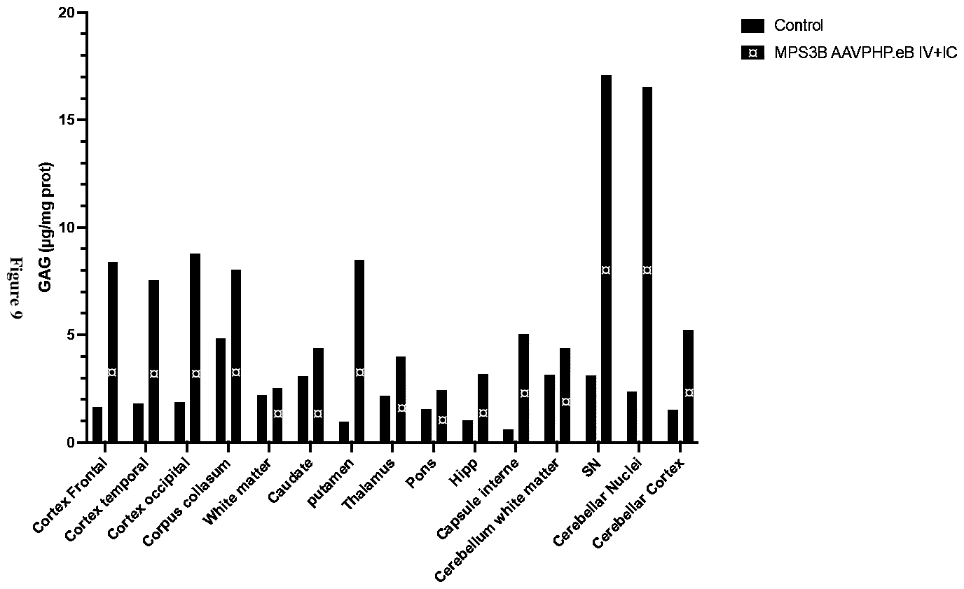

The second crucial point of our study was the evaluation of NAGLU activity in the brain and more largely in the whole CNS, the peripheral organs as in the CSF of injected animals (FIGS. 7 and 8). A significant NAGLU activity was detected in brain regions with an overall restoration of 69% of the activity of a normal dog and up to 121% in the cerebellum, 85% in the spinal cord and 132% in the DRG and 97% in the peripheral nerves (FIG. 7). A 100% rescue was observed in the spleen and only 25% of the normal activity in the liver. In addition, NAGLU activity was clearly increased in the CSF compared to baseline with no activity and at 3 months reach 50% of the control dog.

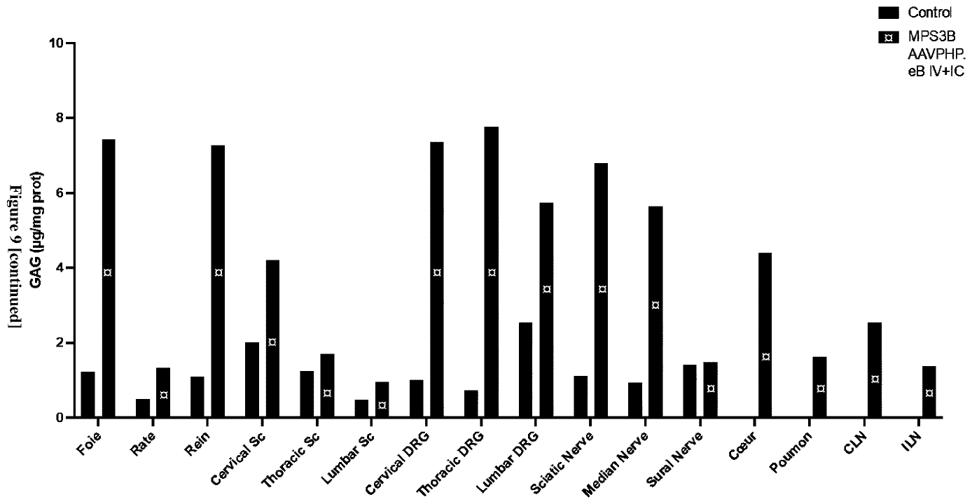

Finally, we quantified GAG content in the treated and wild type animal in CNS and peripheral tissues. In the CNS, GAG amounts vary from one tissue to the others but were almost normalized in treated animal such as in white matter, caudate, pons, lumbar spinal cord (FIG. 9). For a complete comparison we still miss one non treated animal.

Overall these data are really encouraging for MPS3B treatment and application in Humans.

### REFERENCES

Throughout this application, various references describe the state of the art to which this invention pertains. The disclosures of these references are hereby incorporated by reference into the present disclosure.
1. Heron B, Mikaeloff Y, Froissart R, Caridade G, Maire I, Caillaud C, et al. Incidence and Natural History of Mucopolysaccharidosis Type III in France and Comparison With United Kingdom and Greece. Am J Med Genet A (2011) 155A:58-68. doi: 10.1002/ajmg.a.33779.
2. Valstar M J, Bruggenwirth H T, Olmer R, Wevers R A, Verheijen F W, Poorthuis B J, et al. Mucopolysaccharidosis Type IIIB may Predominantly Present With an Attenuated Clinical Phenotype. J Inherit Metab Dis (2010) 33:759-67. doi: 10.1007/s10545-010-9199-y.
3. Weber B, Guo X H, Kleijer W J, van de Kamp J J, Poorthuis B J, Hopwood J J. Sanfilippo Type B Syndrome (Mucopolysaccharidosis III B): Allelic Heterogeneity Corresponds to the Wide Spectrum of Clinical Phenotypes. Eur J Hum Genet (1999) 7:34-44. doi: 10.1038/sj.ejhg.5200242.
4. Beneto N, Vilageliu L, Grinberg D, Canals I. Sanfilippo Syndrome: Molecular Basis, Disease Models and Therapeutic Approaches. Int J Mol Sci (2020) 21:7819-39. doi: 10.3390/ijms21217819.
5. Cheng S H. Gene Therapy for the Neurological Manifestations in Lysosomal Storage Disorders. J Lipid Res (2014) 55:1827-38. doi: 10.1194/jlr.R047175.
6. Mijanovic O, Brankovic A, Borovjagin A, Butnaru D V, Bezrukov E A, Sukhanov R B, et al. Battling Neurodegenerative Diseases With Adeno-Associated Virus-Based Approaches. Viruses (2020) 12:460-85. doi: 10.3390/v12040460.
7. Tardieu M, Zerah M, Husson B, de Bournonville S, Deiva K, Adamsbaum C, et al. Intracerebral Administration of Adeno-Associated Viral Vector Serotype rh.10 Carrying Human SGSH and SUMF1 cDNAs in Children With Mucopolysaccharidosis Type IIIA Disease: Results of a Phase I/II Trial. Hum Gene Ther (2014) 25:506-16. doi: 10.1089/hum.2013.238.
8. Cressant A, Desmaris N, Verot L, Brejot T, Froissart R, Vanier M T, et al. Improved Behavior and Neuropathology in the Mouse Model of Sanfilippo Type IIIB Disease After Adeno-Associated Virus-Mediated Gene Transfer in the Striatum. J Neurosci (2004) 24:10229-39. doi: 10.1523/JNEUROSCI.3558-04.2004.
9. Haurigot V, Marco S, Ribera A, Garcia M, Ruzo A, Villacampa P, et al. Whole Body Correction of Mucopolysaccharidosis IIIA by Intracerebrospinal Fluid Gene Therapy. J Clin Invest (2013) 123:3254-71. doi: 10.1172/JCI66778 10. Ellinwood N M, Wang P, Skeen T, Sharp N J, Cesta M, Decker S, et al. A Model of Mucopolysaccharidosis IIIB (Sanfilippo Syndrome Type IIIB): N-acetylalpha-D-glucosaminidase Deficiency in Schipperke Dogs. J Inherit Metab Dis (2003) 26:489-504. doi: 10.1023/a:1025177411938.
11. Ellinwood N M, Ausseil J, Desmaris N, Bigou S, Liu S, Jens J K, et al. Safe, Efficient, and Reproducible Gene Therapy of the Brain in the Dog Models of Sanfilippo and Hurler Syndromes. Mol Ther (2011) 19:251-9. doi: 10.1038/mt.2010.265.

1. A method for treating Sanfilippo syndrome type B (MPS3B) in a subject in need thereof, comprising administering to the subject a therapeutically effective amount of a vector comprising a full sequence of N-acetylglucosaminidase, alpha (NAGLU) encoding nucleic acid, wherein the vector is administrated to said subject in the venous system and/or directly in the brain.

2. The method according to claim 1, wherein the vector comprises a nucleic acid sequence that encodes the amino acid sequence SEQ ID No 2.

3. The method according to claim 1, wherein the vector comprises the nucleic acid sequence SEQ ID No 1 or a variant thereof.

4. The method according to claim 1, wherein the vector is selected from the group consisting of adenovirus, lentivirus, retrovirus, herpesvirus and Adeno-Associated Virus (AAV) vectors.

5. The method according to claim 1, wherein the vector is an AAV vector.

6. The method according to claim 5, wherein the vector is an AAVPHP.eB. vector.

7. The method according to claim 1, wherein the vector is administered intravenously and intracerebrally.

8. A pharmaceutical composition for use in the treatment of Sanfilippo syndrome type B (MPS3B) in a subject in need thereof which comprises a therapeutically effective amount of a vector as defined in claim 1.

9. (canceled)


SEQUENCE LISTING (2 sequences)

SEQ ID NO: 1 - DNA, 2232 bp, Homo sapiens
  source 1..2232: mol_type = genomic DNA
atggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactctttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattacccccgctgggtggccggctcttggtga

SEQ ID NO: 2 - protein, 743 aa, Homo sapiens
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW